(19) United States
(12) Patent Application Publication
(10) Pub. No.: US 2014/0187885 A1
(43) Pub. Date: Jul. 3, 2014

(54) Sensor for Measuring Vital Parameters in the Auditory Canal

(71) Applicant: Johannes Kreuzer, Munich (DE)

(72) Inventor: Johannes Kreuzer, Munich (DE)

(73) Assignee: Cosinuss GmbH, Munich (DE)

(21) Appl. No.: 14/124,514

(22) PCT Filed: Jun. 7, 2012
(86) PCT No.: PCT/EP2012/060836

§ 371 Date: Dec. 6, 2013

(30) Foreign Application Priority Data
Jun. 7, 2011 (DE) 10 2011 077 066.6
Aug. 30, 2011 (DE) 10 2011 081 815.4

(51) Int. Cl. A61B 5/00; A61B 5/024; A61B 5/1455

(52) U.S. Cl. CPC A61B 5/6817; A61B 5/742; A61B 5/486; A61B 5/14551; A61B 5/02416; A61B 5/741
USPC 600/324; 600/300

(57) ABSTRACT

The invention relates to a sensor for measuring a vital parameter in the auditory canal of a human or of an animal and to a method for determining tissue variables, preferably the oxygen saturation in the blood, particularly for performing pulse oximetry, preferably transmission pulse oximetry, in the auditory canal. The sensor comprises a sensor element support (2), which can be positioned at least partially in the auditory canal (8), a sensor element (1), which is connectable to the sensor element support (2) and can be positioned at least partially in the auditory canal (8), and a positioning element (3) suitable for positioning the sensor element support (2) in the auditory canal (8), wherein the positioning element (3) is connectable at least at one end to the sensor element support (2), wherein the position of the sensor element (1) in the auditory canal (8) is determined by the depth of insertion of the sensor element support (2) in the auditory canal (8), and wherein the positioning element (3) has a suitable shape, simulating the anatomy of the inside of the pinna, and is suitable for pressing the sensor element support (2) into the interior of the auditory canal (8) in such a way that the sensor is held stably and a continuous measurement of the vital parameter takes place in the auditory canal (8). A sensor is thus provided that is able to carry out continuous and exact measurement of vital parameters during movement of the body and that at the same time is cosmetically inconspicuous.

The invention relates to a sensor for measuring a vital parameter in the ear canal of a person or animal. The invention also relates to a process for determining optical tissue parameters, preferably the oxygen saturation in the blood, in particular to perform pulse oximetry in the ear canal, preferably transmission pulse oximetry.

Measurement of important physiological parameters in humans or animals, in particular continuously and under mobile conditions, is indispensable for modern medicine. This includes in particular the measurement of body core temperature or body temperature, optical tissue parameters, such as the arterial oxygen saturation, the heart rate, and the respiration rate, the concentration of specific compounds in the blood and/or tissue, but also the measurement of mechanical parameters, such as orientation, position, and acceleration. The ear canal has proven suitable as monitoring location for such non-invasive measurements.

Many diagnostic and therapeutic measures and many surgical procedures that were earlier performed as stationary care, occur these days mostly on an outpatient basis, or at least with significantly shorter hospitalization periods. A need exists to monitor outpatients more or less on a continual basis, to ensure their health, and at the same time to detect endangerments outside of the hospital. Many hazards occur in daily life, such as for example diabetes, allergies, hypertension, or lipometabolic disorders.

Measurement of the body temperature is normally performed only selectively and discontinuously. In principle, measurement of the body temperature can be made in various more or less appropriate locations, whereby the temperature measured is then more or less relevant to that which is designated as “body (core) temperature”. On the one hand, the body (core) temperature shall be understood to be the central temperature in the spatial-anatomical sense, for example the temperature of the innermost organs, which is not directly disturbed by external influences. On the other hand, it is also understood to be a temperature measured near the hypothalamus, because the physiologic sensor for regulating the temperature in humans is located here, and thus is of primary importance for the thermal control of the organism.

The radiation sensors that are described by the prior art are not very accurate, and measurements of the body temperature on the ear are normally performed only selectively, meaning that a discontinuous measurement occurs. The body temperature is an important physiological parameter for general monitoring, because it reflects many important states of the body.

Pulse oximetry is an optical process for determining the oxygen saturation via photometry of the tissue; it is widespread, and has already been miniaturized. Mobile pulse oximetry normally fails at the measuring point, because the measuring point is almost always a finger. Other measurement locations are less reliable. There is a high demand to make a mobile pulse oximetry available that is non-disruptive in daily life. The devices known from the prior art usually comprise an inconvenient and complex design, which include significant sources of error. These devices usually offer little stability. In addition, the sensor is usually mechanically unstable, and lacks mechanical correspondence to the auricle.

The purpose of the invention is to provide a sensor that is also suitable to monitor mobile individuals who are participating in everyday life, whereby at the same time the sensor shall be cosmetically unobtrusive, and frequently or continuously measures vital parameters. In addition, the possibility of determining optical tissue parameters in the ear canal should be made available. Here, the term “continuously” means that the frequency of measurements is high enough that changes in the measured values are sampled with a frequency that is sufficient for all diagnostic and/or therapeutic purposes, so that a quasi-continuous determination is possible. At the same time, the mobile sensor should be dimensioned for power consumption so that acceptable operating times are obtained with available energy cells.

This purpose is solved in that a sensor is provided for measuring a vital parameter in the ear canal of a person or animal, whereby the sensor comprises a sensor element fixture that can be positioned at least partially in the ear canal, a sensor element, what can be connected to the sensor element fixture, and can be positioned at least partially in the ear canal, and a positioning element, suitable to position the sensor element fixture in the ear canal, whereby the positioning element can be connected to the sensor element fixture on at least one end, whereby the position of the sensor element in the ear canal will be determined by the insertion depth of the sensor element fixture in the ear canal, and wherein the positioning element exhibits a suitable form, corresponding to the anatomy of the interior face of the auricle, and is suitable to press the sensor element fixture into the interior of the ear canal, in such a manner that the sensor will be held stabile, and a continuous measurement of the vital parameters occurs in the ear canal.

The outer ear canal is used preferably as measuring location, which is well suited for mobile or continuous sensors on the one hand through its combination of physiological characteristics, and on the other hand by mechanical, technical, and/or functional characteristics. The expression that a vital parameter is measured means that at least one, but also two or more parameters may be measured simultaneously. The term that the sensor element can be connected with the sensor element fixture means that the sensor element may be connected to the sensor element fixture, in particular through other components of the sensor or also through tubes, wires, cables, or similar. The sensor element may also be in direct contact with the sensor element fixture, meaning it is attached directly to the sensor element attachment. The sensor element preferably comprises the sensor element fixture, meaning that the sensor element fixture is formed as a part of the sensor element. The expression that the position of the sensor element in the ear canal is determined by the insertion depth of the sensor element fixture in the ear canal means that the positioning of the sensor element occurs relative to the sensor element fixture, so that a precise and continuous measurement of the vital parameter may occur, in particular the sensor element and the sensor element fixture are positioned near to one another, so that signal transmission between the sensor element fixture and the sensor element can function correctly. The positioning element and sensor element fixture are preferably formed as a single piece.

The term “sensor element” shall be understood to mean the sensor, thus the actual measurement probe, which converts the actual physiologic or biologic parameters in an electrical signal, for example a converter, a semiconductor, a measurement probe, or electrodes. The term “sensor element fixture” and “positioning element” are understood as functional and/or mechanical connective working devices, which together position the sensor element relative to the ear canal wall in such a manner, that the physiologic or biologic parameter may be measured with as little as possible disruption. The purpose of the sensor element fixture is to define the radial position of the sensor element, whereas the purpose of the positioning element is to define the axial position. In accordance with other preferred sample embodiments of the invention, both functions can neither be separated nor merged with one another. The term “sensor” stands in particular for the combination of sensor, sensor element fixture, and positioning elements. In accordance with other preferred sample embodiments of the invention, the term “sensor” also means the evaluation unit, which is preferably located behind the ear, and is additionally suitable to provide the sensor with stability.

According to one preferred sample embodiment of the invention, the sensor comprises a stabilizing element, for example a retaining thread, which is suitable to retain the sensor inside the auricle, and can be at least partially attached to the positioning element and/or the sensor element fixture. Preferably, the stabilization element can be at least partially connected to a spring, wherein the spring is also suitable to press the stabilizing element against the auricle.

In accordance with another preferred sample embodiment of the invention, the sensor element fixture is formed elastically in such a manner, so that through its spring force, it exerts a defined pressure on the wall of the ear canal, so that it adapts its form to the form of the ear canal.

In accordance with another preferred sample embodiment of the invention, the sensor comprises a control unit to monitor the positioning of the sensor in the ear canal, wherein the position of the sensor element and/or of the sensor element fixture will be determined on the basis of the temporal change of the measurement signal during continuous measurement of the vital parameter, and after a change in the measurement signal of a predetermined value, the difference between the current position from the desired position will be indicated via a display unit.

In accordance with another preferred sample embodiment of the invention, the sensor comprises a control unit to monitor the positioning of the sensor in the ear canal, wherein the control unit determines the optimum position of the sensor, and can indicate this via the display unit.

Preferably an evaluation unit is provided, which is positioned on the auricle at the opposite end of the positioning element from the sensor element, and is suitable to hold the sensor stable. The evaluation unit preferably sends the information obtained subsequent to digitization of the analog measurement levels to further peripheral units, in particular to a mobile radio unit and/or a computer. The sensor preferably comprises a speaker, which is suitable to return biofeedback about the measured vital parameters within the ear back into the interior of the ear canal. The measured vital parameters, such as pulse rate, can also be used advantageously to indicate to the person for example whether he or she should run faster or slower during jogging, according to whether the measured pulse is too high or too low. Music or other acoustic waves are transmitted preferably simultaneously, in particular in the range between 0 and 20 kHz.

In accordance with another preferred sample embodiment of the invention, the sensor element fixture and the sensor element are designed in the form of a light emitter and a light receiver, wherein the sensor additionally comprises a transmission means, which is suitable to prevent light directly reaching the light receiver from the light emitter without trans-illuminating the tissue. If light reaches the light receiver in a direct path from the light emitter, the expert speaks of “shunt light”. “Shunt light” means that light reaches the light receiver in a direct path from the light emitter, without having trans-illuminated blood-filled tissue along its way. In the case of transmission pulse oximetry, no shunt light normally occurs, because no light reaches the receiver that has not penetrated the blood-filled tissue. The transmission means comprises preferably a disk-formed or a screen-formed element, wherein the light emitter is arranged on one side and the light receiver on the other side, and/or the transmission means comprises a screen-formed double sensor element fixture, in which the light emitter is provided on the first screen and the light receiver on the second screen. The sensor element fixture is preferably also suitable to maximize the length of the light path or suppress contracted light paths, towards the light-reducing, particularly preferably opaque coverage of more than half of half of the circumference of the inner ear canal in the scope of the net light path toward both sides of the light receiver.

Here the expert understands “net light path” to mean the shortest light path that light can take from the exit of the light emitter to the entrance of the light receiver, wherein dispersion and refraction effects in the vital tissues are considered. An unlimited multitude of propagation paths of various lengths depending on the multiple dispersion centers are possible along the path from the light emitter to the light receiver, wherein beams of light may be bent, refracted, or dispersed along each path. The “net light path” represents the light path with the greatest light intensity at the light receiver, whereby dispersion and refraction effects are considered.

In accordance with another preferred sample embodiment of the invention, the sensor is designed with an adjustable size, the sensor element fixture and/or the positioning elements is/are interchangeable and/or designed with adjustable size. This contributes toward easy exchangeability of the individual components and/or adaptation to the individual anatomical conditions of the person or animal, in particular also for reasons of hygiene.

The sensor element fixture is preferably curved and/or formed as a screen; particularly preferably, the sensor element fixture comprises silicone as material.

The vital parameters preferably comprise at least one physiologic, biochemical, and/or bioelectric parameter, in particular the vital parameter is selected from the group comprised of body temperature, oxygen saturation of the blood, heart rate, an electrical heart parameter, the respiration rate, the concentration of substances dissolved in the blood, in particular the arterial oxygen saturation, the concentration of substances present in the tissue, physical activity, and the body's orientation.

In accordance with another aspect of the invention, the purpose is thus solved in that a process is provided for determining optical tissue parameters in the ear canal, preferably the oxygen saturation in the blood, in particular to perform pulse oximetry, preferably transmission pulse oximetry, wherein the process comprises the step: the optimization of the auxiliary dimension Ω for the determination of oxygen saturation, preferably to a value of ≦0.5, particularly preferably to a value ≧0.4 and ≦0.5, especially particularly preferably to a value of approximately 0.45, for human oxihemoglobin and/or for a wavelength, preferably of 740 nm and/or 880 nm, or for symmetric spectra of light-emitting diodes of the same central wavelength.

The range ≧0.4 and ≦0.5 produces advantageously that here the same values result mathematically and technically measured. Thus, measurement errors are in particular reduced and the measurement is more precise.

The process is preferably conducted with the introduction of acoustic waves, in particular in the range between 0 and 20 kHz.

Preferably, at least two wavelengths are selected between 660 nm and 1000 nm, particularly preferably wavelengths around 740 nm and/or around 880 nm, especially particularly preferably one wavelength 810 nm and one wavelength >810 nm. The wavelength or the wavelength range will be advantageously so selected, so that in particular a large separation exists between the absorption coefficients and/or the spectral absorption curve is level in this region. Thus, the sensitivity to disruption will also be advantageously reduced and the measurement range expanded.

The modulation depth MD for monochromatic light or for the light spectrum results from division of the alternating light spectrum, also designated as AC, by the direct light spectrum, also designated as DC:

$${MD}_{λ} = \frac{{AC}_{λ}}{{DC}_{λ}} .$$

Preferably, pulse oximetry permits determination of the pulse rate and/or the respiration rate, among others. Other optical tissue parameters may also be determined. If two or more spectral ranges are used, and the respective modulation depths are each set in relationship to one another for corresponding wavelengths, each respective modulation depth thus produces a variable Ω, which in English is also designated as the ratio:

$$Ω = \frac{{MD}_{{λ}_{1}}}{{MD}_{{λ}_{2}}}$$

$$and$$

$$Ω = \frac{{( \frac{AC}{DC} )}_{{λ}_{1}}}{{( \frac{AC}{DC} )}_{{λ}_{2}}} .$$

Using these values permits preferably the arterial oxygen saturation, the venous oxygen saturation, and/or the arterial-venous saturation difference to be determined.

Selecting the outer ear canal as a measuring location is convenient for many reasons, for example, wearing comfort and wearing appearance are very favorable for the outer ear canal, and movements of the head are rather limited and less frequent compared to the extremities. The outer ear canal is also well supplied with blood, which is suitable for optical plethysmographic measurement principles, such as the pulse oximetry. Preferably, the sound will reach the eardrum largely unimpeded by the various components of the sensor. This occurs preferably in that the various components of the sensor dampen the sound minimally or non-significantly, and/or that the sensor element fixture is constructed so that it either allows the sound propagation in the direction of the eardrum, meaning it is not perceptibly hindered, or it is even actively promoted. Preferably, the sensor element fixture is provided with suitable holes and/or cavities and/or channels that ensure that the outer ear canal is not completely closed or dampened.

Preferably, the positioning element defines the axial position of the sensor element in the ear canal, meaning its depth in the ear canal, which represents a parameter appropriate for the measurement. The positioning element advantageously serves to retain the sensor, meaning it is responsible for the mechanical stability of the sensor. Preferably, the evaluation unit behind the ear is held against the head by clamping by means of a suitable clamping means. All components are preferably colored in skin tones or transparent, so that the sensor is especially inconspicuous. The unit preferably comprises the energy cells as well as a portion of the electronics for signal processing.

In accordance with another preferred sample embodiment of the invention, the sensor element is located outside the ear canal. Here the physical measurement levels obtained in the ear canal are combined with the sensor element outside the ear canal. It is thus an idea of the invention to be independent of the arrangement of the optical components relative to one another.

The invention will be explained in further detail on the basis of a preferred sample embodiment with reference to the figures as follows:

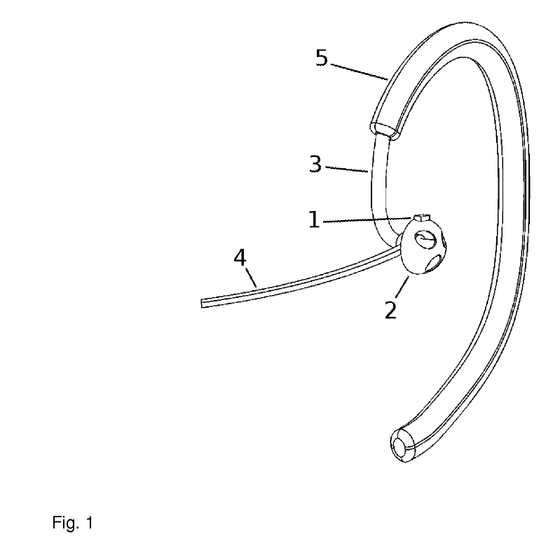

FIG. 1 shows the sensor in accordance with a first preferred sample embodiment of the invention;

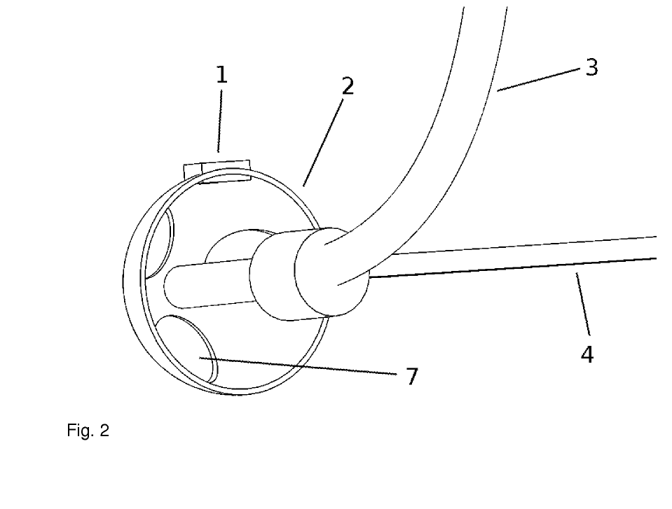

FIG. 2 shows a sensor element fixture in accordance with the first preferred sample embodiment of the invention;

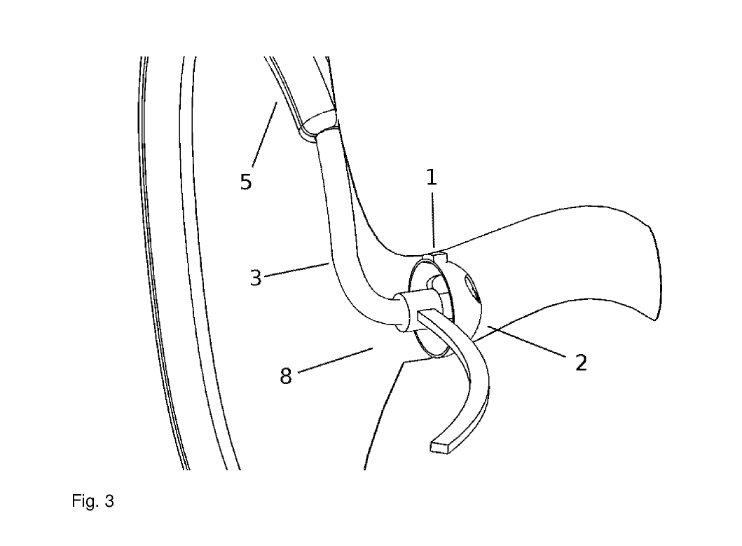

FIG. 3 shows a cross-section of the outer ear canal with the sensor element on a simple sensor element fixture in accordance with a second preferred sample embodiment of the invention;

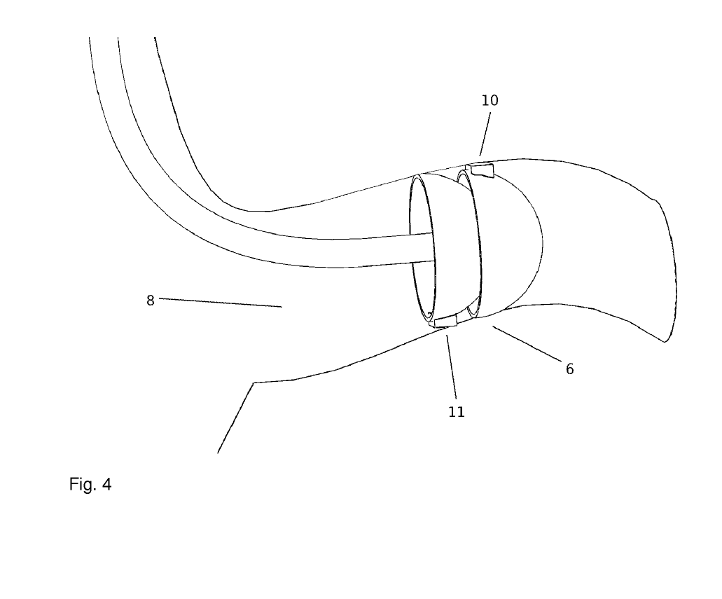

FIG. 4 shows a cross-section of the outer ear canal with the sensor element on a double sensor element fixture in accordance with a third preferred sample embodiment of the invention;

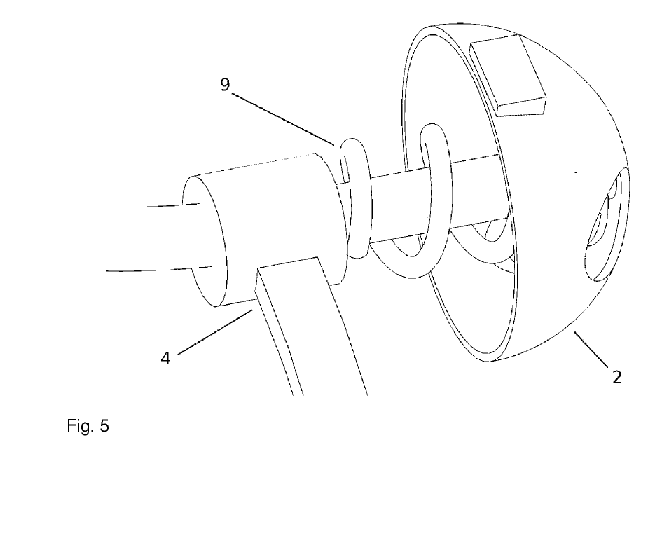

FIG. 5 shows a view of a stabilizing element with a spring that generates stable positioning through pressure on the auricle, in accordance with a fourth preferred sample embodiment of the invention;

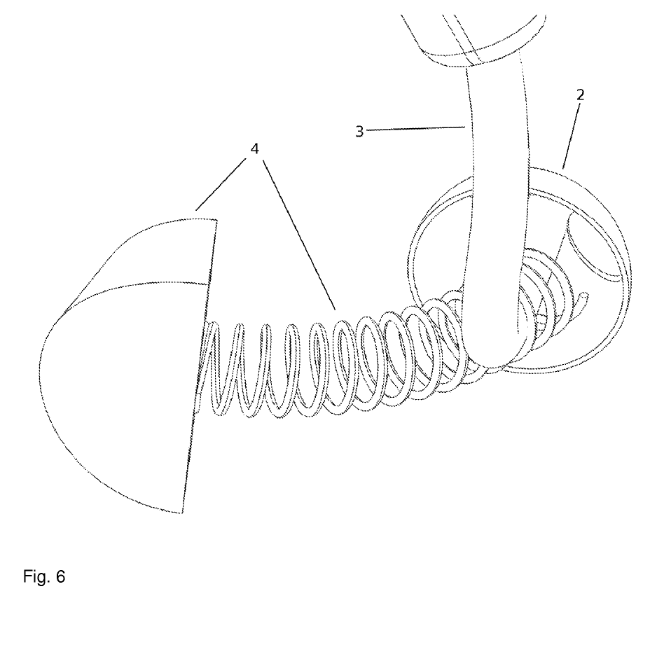

FIG. 6 shows a view of an additional stabilizing element that can be positioned on the interior surface of the auricle, and contributes to retaining the sensor, in accordance with a fifth preferred sample embodiment of the invention;

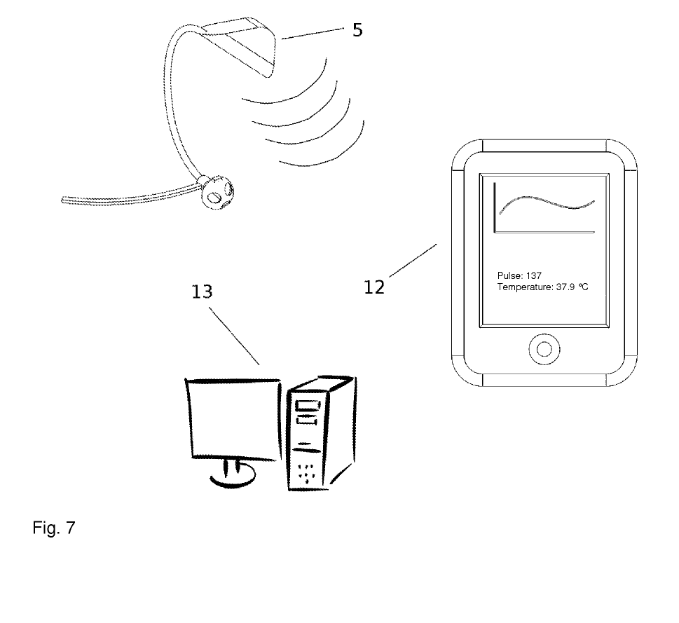

FIG. 7 shows a schematic representation of the data communication from the sensor to a mobile radio device or a computer, in accordance with a sixth preferred sample embodiment of the invention.

FIG. 1 shows a sensor in accordance with a first preferred sample embodiment of the invention. From FIG. 1 it can be recognized that an evaluation unit 5 behind the ear is connected to the sensor element fixture 2 through the anatomically adjusted, preformed positioning element 3. A stabilizing element 4 serves additionally to retain the sensor in the auricle. The sensor element 1, here a temperature sensor, is fastened onto or into the sensor element fixture 2. An energy cell, evaluation electronics, as well as a transmitter are located in the evaluation unit 5.

FIG. 2 shows the sensor element fixture 2 from FIG. 1 in accordance with the first preferred sample embodiment of the invention. The sensor element fixture 2 comprises cavities in order not to hinder sound propagation. The sensor element 1, here a temperature sensor, is fastened onto the sensor element fixture 2. The sensor element fixture 2 and positioning element 3 are mechanically connected to each other.

FIG. 3 shows a cross-section of the outer ear canal 8 and a part of the sensor as example of a temperature sensor in accordance with a second preferred sample embodiment of the invention. The sensor element fixture 2 is mechanically connected to the positioning element 3, wherein the sensor element fixture 2 holds the sensor element 1. The sensor elements 1 come to lie upon the skin and are located in the outer ear canal 8 in front of the eardrum.

FIG. 4 shows a cross section through the outer ear canal 8 and the components of the positioning element 3 located in the ear canal 8, the double sensor element fixture 6, light emitter 10, and light receiver 11, in accordance with a third preferred sample embodiment of the invention. This is in particular suitable for plethysmographic sensors, for example, pulse oximetry in the outer ear canal 8. It can be recognized that an optical separation of the two sensor elements is 10 and 11 by the double sensor element fixture 6 in order to prevent shunt light occurs, meaning that because of the optical separation by the double sensor element fixture 6, the light emitter 10 cannot directly reach the light receiver 11, meaning by circumventing a light path within the tissue. In addition, one recognizes that the light emitter 10 and light receiver 11 are pressed onto the skin of the ear canal 8 opposite to one another in axial direction, whereby a lengthening of the light path and the net light path will be achieved. Furthermore, the light emitter 10 and light receiver 11 are offset against one another by about 180° in the radial direction.

FIG. 5 shows a stabilizing element 4, which is pressed outwards by a spring 9 against the inside of the auricle. This simplifies positioning the sensor element fixture 2 in the ear canal.

FIG. 6 shows a stabilizing element 4 in accordance with an additional sample embodiment of the invention. The positioning element 3 connected to the sensor element fixture 2 is stabilized and held in position in the ear canal by the stabilization element 4.

FIG. 7 shows the design of the sensor, comprising the components described in detail in FIG. 1, in accordance with a sixth preferred sample embodiment of the invention. Here wireless or wire-connected units 12 and 13 can be recognized in the form of modules. The evaluation unit 5 is located behind the ear. In addition, one recognizes a display and evaluation unit 13 and a mobile radio unit 12, which may be spatially separated units, arbitrarily merged, or may also be omitted entirely.

1. A sensor for measuring a vital parameter in the ear canal (8) of a person or animal, comprising:
a sensor element fixture (2), which can be positioned at least partially in the ear canal (8),
a sensor element (1), which can be connected to the sensor element fixture (2), and can be positioned at least partially in the ear canal (8), and
a positioning element (3), suitable for positioning the sensor element fixture (2) in the ear canal (8), whereby the positioning element (3) can be connected to the sensor element fixture (2) on at least one end,
wherein the position of the sensor element (1) in the ear canal (8) is determined by the insertion depth of the sensor element fixture (2) into the ear canal (8),
wherein the positioning element (3) exhibits a suitable form, corresponding to the anatomy of the interior face of the auricle, and is suitable for pressing the sensor element fixture (2) into the interior of the ear canal (8) in such a manner, that the sensor is held stable, and a continuous measurement of the vital parameter occurs in the ear canal (8), and
wherein the sensor is designed with an adjustable size, preferably the sensor element fixture (2) and/or the positioning element (3) are designed to be exchangeable and/or adjustable in size.

2. The sensor according to claim 1, comprising a stabilizing element (4), which is suitable for retaining the sensor in the auricle, and can be attached at least partially to the positioning element (3) and/or to the sensor element fixture (2).

3. The sensor according to claim 2, wherein the stabilizing element (4) can be connected at least partially to a spring (9), wherein in addition the spring (9) is suitable for pressing the stabilizing element (4) against the auricle.

4. The sensor according to claim 1, wherein the sensor element fixture (2) is formed elastically in such a manner, so that through its spring force, it exerts a defined pressure on the wall of the ear canal (8), so that it adapts its form to the form of the ear canal (8).

5. The sensor according to claim 1, comprising a control unit for monitoring the positioning of the sensor in the ear canal (8), wherein the position of the sensor element (1) and/or of the sensor element fixture (2) will be determined on the basis of the temporal change of the measurement signal during continuous measurement of the vital parameter, and after a change in the measurement signal by a predetermined value, the difference between the current position from the desired position will be indicated via a display unit.

6. The sensor according to claim 1, comprising a control unit for monitoring the positioning of the sensor in the ear canal (8), wherein the optimum position of the sensor can be indicated by a display unit.

7. The sensor according to claim 1, wherein an evaluation unit (5) is provided that can be positioned on the auricle on an end of the positioning element (3) opposite to the sensor element (1), and is suitable for holding the sensor stable.

8. The sensor according to claim 1, comprising a speaker that is suitable for returning biofeedback about the vital parameters measured in the ear canal (8) back to the interior of the ear canal (8).

9-10. (canceled)

11. A sensor for measuring a vital parameter in the ear canal (8) of a person or animal, comprising:
a sensor element fixture (2), which can be positioned at least partially in the ear canal (8), wherein the sensor element fixture (2) is formed elastically in such a manner, so that through its spring force, it exerts a defined pressure on the wall of the ear canal (8), so that it adapts its form to the form of the ear canal (8);
a sensor element (1), which can be connected to the sensor element fixture (2), and can be positioned at least partially in the ear canal (8);
a positioning element (3), suitable for positioning the sensor element fixture (2) in the ear canal (8), whereby the positioning element (3) can be connected to the sensor element fixture (2) on at least one end; and
a speaker that is suitable for returning biofeedback about the vital parameters measured in the ear canal (8) back to the interior of the ear canal (8);
wherein the position of the sensor element (1) in the ear canal (8) is determined by the insertion depth of the sensor element fixture (2) into the ear canal (8);
wherein the positioning element (3) exhibits a suitable form, corresponding to the anatomy of the interior face of the auricle, and is suitable for pressing the sensor element fixture (2) into the interior of the ear canal (8) in such a manner, that the sensor is held stable, and a continuous measurement of the vital parameter occurs in the ear canal (8); and
wherein the sensor is designed with an adjustable size, preferably the sensor element fixture (2) and/or the positioning element (3) are designed to be exchangeable and/or adjustable in size.

12. The sensor according to claim 11, comprising a stabilizing element (4), which is suitable for retaining the sensor in the auricle, and can be attached at least partially to the positioning element (3) and/or to the sensor element fixture (2).

13. The sensor according to claim 12, wherein the stabilizing element (4) can be connected at least partially to a spring (9), wherein in addition the spring (9) is suitable for pressing the stabilizing element (4) against the auricle.

14. The sensor according to claim 11, comprising a control unit for monitoring the positioning of the sensor in the ear canal (8), wherein the position of the sensor element (1) and/or of the sensor element fixture (2) will be determined on the basis of the temporal change of the measurement signal during continuous measurement of the vital parameter, and after a change in the measurement signal by a predetermined value, the difference between the current position from the desired position will be indicated via a display unit.

15. The sensor according to claim 11, comprising a control unit for monitoring the positioning of the sensor in the ear canal (8), wherein the optimum position of the sensor can be indicated by a display unit.

16. The sensor according to claim 11, wherein an evaluation unit (5) is provided that can be positioned on the auricle on an end of the positioning element (3) opposite to the sensor element (1), and is suitable for holding the sensor stable.

17. A process for determining optical tissue parameters, preferably the oxygen saturation in the blood, in particular for performing pulse oximetry, preferably transmission pulse oximetry in the ear canal (8), comprising the step of:
providing a sensor having (a) a sensor element fixture (2), which can be positioned at least partially in the ear canal (8), (b) a sensor element (1), which can be connected to the sensor element fixture (2) and can be positioned at least partially in the ear canal (8), (c) a positioning element (3), suitable for positioning the sensor element fixture (2) in the ear canal (8), whereby the positioning element (3) can be connected to the sensor element fixture (2) on at least one end, (d) wherein the position of the sensor element (1) in the ear canal (8) is determined by the insertion depth of the sensor element fixture (2) into the ear canal (8), (e) wherein the positioning element (3) exhibits a suitable form, corresponding to the anatomy of the interior face of the auricle, and is suitable for pressing the sensor element fixture (2) into the interior of the ear canal (8) in such a manner, that the sensor is held stable, and a continuous measurement of the vital parameter occurs in the ear canal (8), and (f) wherein the sensor is designed with an adjustable size, preferably the sensor element fixture (2) and/or the positioning element (3) are designed to be exchangeable and/or adjustable in size; and
optimizing the auxiliary dimension Ω for the determination of oxygen saturation, preferably to a value of ≦0.5, particularly preferably to a value ≧0.4 and ≦0.5, especially particularly preferably to a value of approximately 0.45, for human oxihemoglobin and/or for a wavelength, preferably of 740 nm and/or 880 nm, or for symmetric spectra of light-emitting diodes of the same central wavelength.

18. The method according to claim 17, comprising a stabilizing element (4), which is suitable for retaining the sensor in the auricle, and can be attached at least partially to the positioning element (3) and/or to the sensor element fixture (2).

19. The method according to claim 18, wherein the stabilizing element (4) can be connected at least partially to a spring (9), wherein in addition the spring (9) is suitable for pressing the stabilizing element (4) against the auricle.

20. The method according to claim 17, wherein the sensor element fixture (2) is formed elastically in such a manner, so that through its spring force, it exerts a defined pressure on the wall of the ear canal (8), so that it adapts its form to the form of the ear canal (8).

21. The method according to claim 17, comprising a control unit for monitoring the positioning of the sensor in the ear canal (8), wherein the position of the sensor element (1) and/or of the sensor element fixture (2) will be determined on the basis of the temporal change of the measurement signal during continuous measurement of the vital parameter, and after a change in the measurement signal by a predetermined value, the difference between the current position from the desired position will be indicated via a display unit.

22. The method according to claim 17, comprising a speaker that is suitable for returning biofeedback about the vital parameters measured in the ear canal (8) back to the interior of the ear canal (8).
